(19) United States
(12) Patent Application Publication
(10) Pub. No.: US 2020/0164004 A1
(43) Pub. Date: May 28, 2020

(54) BACTERIAL COMPOSITION AND/OR DERIVATIVES THEREOF WHOSE BIOLOGICAL ACTIVITY HAS BEEN SPECIFICALLY STUDIED FOR THE IMPROVEMENT OF THE STATE OF HEALTH DIFFERENTIATED FOR MALES AND FEMALES

(71) Applicant: PROBIOTICAL S.p.A., Novara (IT)

(72) Inventor: Giovanni MOGNA, Novara (IT)

(21) Appl. No.: 16/624,249

(22) PCT Filed: Jun. 19, 2018
(86) PCT No.: PCT/IB2018/054508

§ 371 Date: Dec. 18, 2019

(30) Foreign Application Priority Data
Jun. 19, 2017 (IT) 102017000068000

(51) Int. Cl. A61K 35/745; A61K 35/747; A61P 25/16; A61K 9/00

(52) U.S. Cl. CPC A61K 35/745; A61K 9/0053; A61P 25/16; A61K 35/747

(57) ABSTRACT

The present invention relates to a bacterial composition and/or derivatives thereof whose biological activity has been specifically studied for the improvement of the state of health differentiated for male and female human subjects.

The present invention relates to a bacterial composition and/or derivatives thereof whose biological activity has been specifically studied for the improvement of the state of health differentiated for male and female human subjects. In particular, the present invention relates to a composition comprising at least one selected bacterial strain and/or derivatives thereof whose biological activity has been specifically studied for the improvement of the state of health differentiated for male and female human subjects, said subjects being affected, by way of non-limiting example, by a pathology due to an imbalance in the intestinal microbiota, or a neurodegenerative or cardiovascular pathology, or a pathology linked to an immune system deficit, or a pathology linked to biological processes of physical aging and aging of the skin or cutis, or a pathology linked to biological aging processes which lead to a progressive loss of memory and/or of the ability to concentrate, or a pathology caused by oxidative stress, or an autoimmune pathology, or an inflammatory pathology, or a pathology due to an altered intestinal permeability, and others.

It is well known that in the past twenty years, microorganisms have had a growing development and use in the field of human health. Indeed, to date a very substantial amount of scientific literature has been developed, in which many bacterial strains, including probiotic ones, have been first isolated and morphologically characterised and then studied in order to understand their characteristics and biological system, as well as their ability to interact with the human body in various regions.

The aim of the research conducted up to today has always been oriented towards developing specific finished products based on bacterial strains such as, for example, dietary supplements or medical devices or pharmaceutical compositions and cosmetic compositions, all of which intended indistinctly for a generalised use by human subjects, be they male or female, having a same distinct need.

However, it is also known that the use of bacterial strains is not always capable of bringing about an improvement in the health conditions of a human subject in need, since the human body is particular and differs from one subject to another.

The Applicant has addressed the technical problem of improving, given the same specific pathology or disorder, the state of health of a human subject in need. In particular, the Applicant has addressed the technical problem of maximising, given the same specific pathology or disorder, the effectiveness of one or more bacterial strains.

Following an extensive and in-depth activity of research and development, the Applicant has succeeded in providing a suitable response to the above-mentioned technical problem.

The applicant has invested abundant research activity, both to study the biological activity of many bacterial strains and/or derivatives thereof, and to understand the way in which they interact with the human biological system.

Surprisingly, the Applicant has found that the biological activity of bacterial strains (biotypes) and/or derivatives thereof, belonging to a given genus and species, interact in a targeted and differentiated manner with the biological system of a male or female human subject (male gender or female gender), the pathological condition being equal.

The applicant has found that within the same genus and/or species and/or biotype, bacterial strains have a biological activity capable of improving the state of health in a manner that is differentiated by human gender: male sex and female sex.

Therefore, within the same genus, the same genus and species, the same genus and species and biotype, the Applicant has surprisingly found that there are bacterial strains and/or derivatives thereof that perform better in humans in health terms for the male gender and others that perform in better in health terms for the female gender.

The subject matter of the present invention relates to a composition having the features as defined in the appended claims.

Preferred embodiments of the present invention will emerge clearly from the detailed description that follows and are specified in the appended claims.

In the accompanying Figures, the bacterial strains are indicated by codes with reference to Table 1.

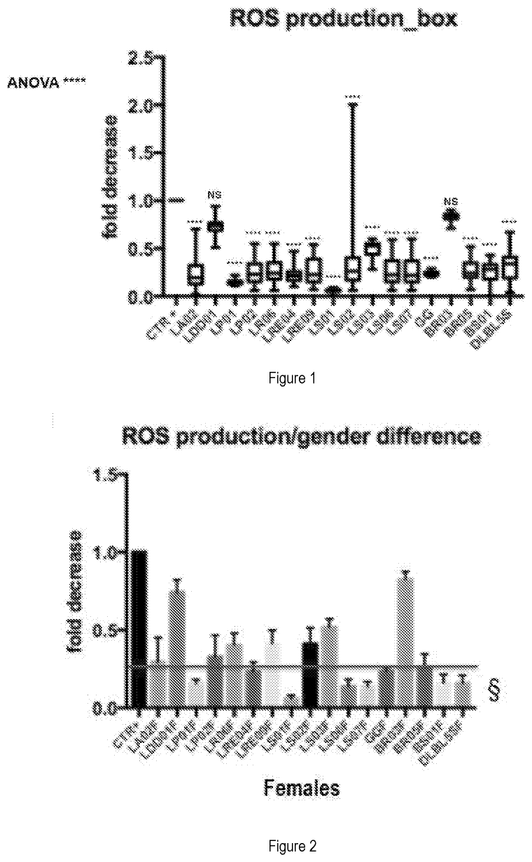

FIG. 1 shows the effect of the bacterial strains in Table 1 on the production of the superoxide anion for the assessment of oxidative stress in subjects affected by Parkinson's disease.

FIG. 2 shows the data of FIG. 1 for female human subjects.

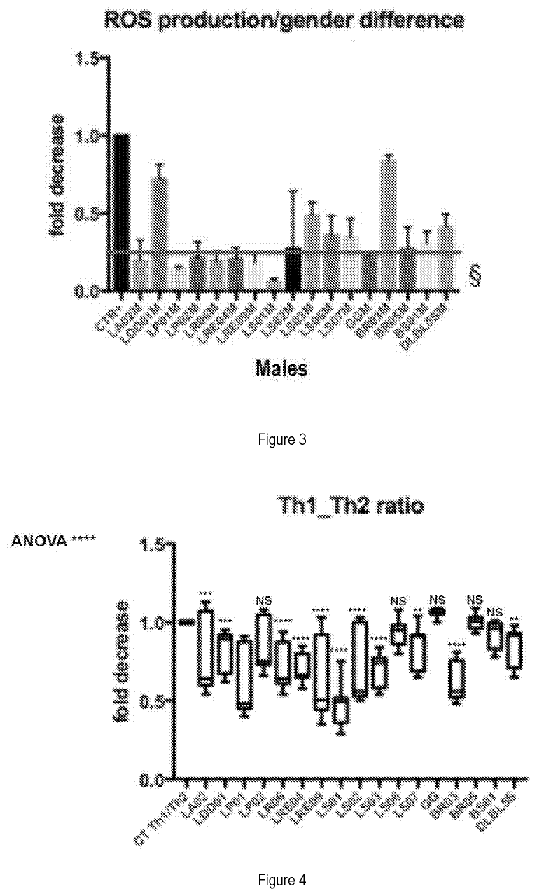

FIG. 3 shows the data of FIG. 1 for male human subjects.

FIG. 4 relates to the activity of modulation of bacterial strains on the Th1 (pro-inflammatory response)/Th2 (anti-inflammatory response) ratio in subjects affected by Parkinson's disease. A decrease in the Th1/Th2 ratio indicates a modulation in an anti-inflammatory direction.

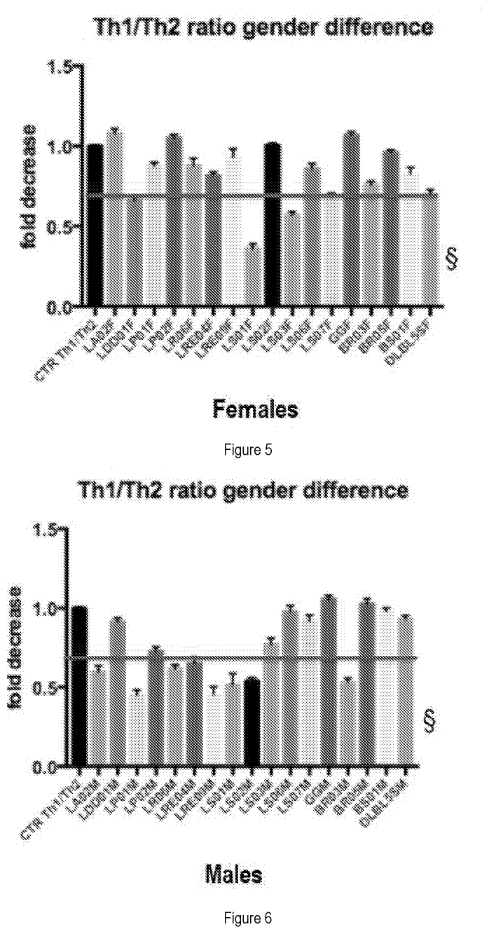

FIG. 5 shows the data of FIG. 4 for female human subjects.

FIG. 6 shows the data of FIG. 4 for male human subjects.

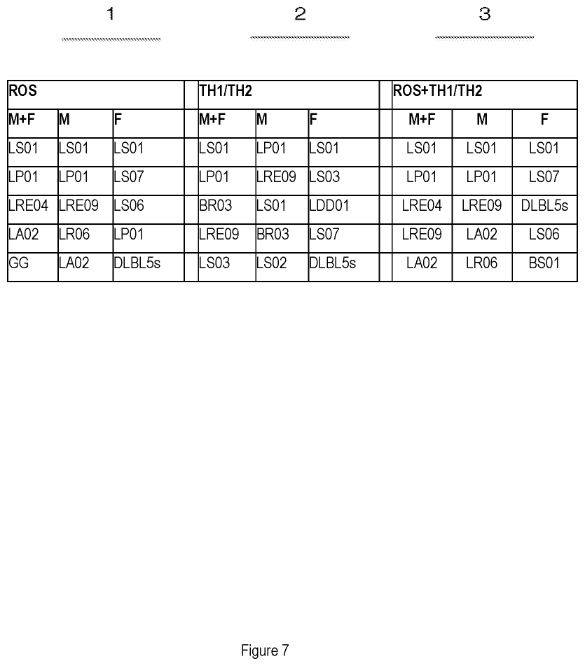

FIG. 7 shows the analysis of the results and the selection of the strains with a greater effect on the production of the superoxide anion for the assessment of oxidative stress and on the anti-inflammatory effect, differentiated for the male gender and the female gender.

In the context of the present invention, “bacterial strains” and/or the “derivatives” thereof are claimed. “Bacterial strains” is meant to include: a) viable bacterial strains and b) dead bacterial strains. The group of dead bacterial strains (b) is meant to include, by way of non-limiting example: b1) bacterial cell wall extracts, b2) tyndallized bacteria, b3) sonicated bacteria (bacteria treated with a sonication process), and b4) lysed bacteria.

“Derivatives” is meant to include, by way of non-limiting example, bioactive peptides derived from a bacterial strain (bacterial metabolites).

In the context of the present invention, the term “composition(s)” is meant to include dietary supplements or the compositions for medical devices or pharmaceutical compositions or cosmetic compositions.

The subject matter of the present invention relates to a composition comprising a mixture which comprises, or consists of, an effective amount of at least one bacterial strain and/or derivatives thereof for use in improving the state of health, differentiated by human gender: male and female.

The subject matter of the present invention relates to a composition comprising a mixture which comprises or, alternatively, consists of an effective amount of at least one bacterial strain and/or derivatives thereof for use in improving the state of health, differentiated for male and female human subjects, said subjects being affected, by way of non-limiting example, by a pathology due to an imbalance in the intestinal microbiota, or a neurodegenerative or cardiovascular pathology, or a pathology linked to an immune system deficit, or a pathology linked to biological processes of physical aging and aging of the skin or cutis, or a pathology linked to biological aging processes which lead to a progressive loss of memory and/or of the ability to concentrate, or a pathology caused by oxidative stress, or an autoimmune pathology, or an inflammatory pathology, or a pathology due to an altered intestinal permeability and others; wherein said at least one bacterial strain and/or derivatives thereof can belong to the genus Lactobacillus, Bifidobacterium, Lactococcus and Streptococcus, and wherein said bacterial strain performs a targeted and differentiated activity in the treatment of a male human subject and a female human subject.

The subject matter of the present invention relates to a composition comprising a mixture which comprises or, alternatively, consists of an effective amount of at least one bacterial strain and/or at least one derivative thereof for use in a method for the curative and/or preventive treatment, in a subject, of the symptoms and/or disorders connected to at least one pathology selected in the group comprising or, alternatively, consisting of (i) a pathology due to an imbalance in the intestinal microbiota, (ii) a neurodegenerative pathology, (iii) a cardiovascular pathology, (iv) a pathology linked to an immune system deficit, (v) a pathology linked to biological processes of physical aging and aging of the skin or cutis, (vi) a pathology linked to biological aging processes which lead to a progressive loss of memory and/or of the ability to concentrate, (vii) a pathology caused by oxidative stress, (viii) an autoimmune pathology, (ix) an inflammatory pathology and (x) a pathology due to an altered intestinal permeability, wherein said at least one bacterial strain belongs to the genus selected in the group comprising or, alternatively, consisting of Lactobacillus, Bifidobacterium, Lactococcus and Streptococcus, wherein the bacterial strain performs a targeted and differentiated activity in the method of treatment for a male human subject and/or a female human subject, wherein said composition is for use in a method of treatment for a male human subject or, alternatively, of a female human subject and wherein said subjects are affected by a pathology selected in the group comprising or, alternatively, consisting of (i) to (x).

The subject matter of the present invention relates to a method for the curative and/or preventive treatment, in a subject, of the symptoms and/or disorders connected to at least one pathology selected in the group comprising or, alternatively, consisting of (i) a pathology due to an imbalance in the intestinal microbiota, (ii) a neurodegenerative pathology, (iii) a cardiovascular pathology, (iv) a pathology linked to an immune system deficit, (v) a pathology linked to biological processes of physical aging and aging of the skin or cutis, (vi) a pathology linked to biological aging processes which lead to a progressive loss of memory and/or of the ability to concentrate, (vii) a pathology caused by oxidative stress, (viii) an autoimmune pathology, (ix) an inflammatory pathology and (x) a pathology due to an altered intestinal permeability, wherein said method of treatment comprises the administration of a composition according to the invention, wherein the at least one bacterial strain of the composition of the invention performs a targeted and differentiated activity in the method of treatment for a male human subject and/or a female human subject, wherein said method of treatment comprises the administration of said composition to a male human subject or, alternatively, a female human subject and wherein said subjects are affected by a pathology selected in the group comprising or, alternatively, consisting of (i) to (x).

Specifically, there is provided a treatment which, for the same pathology, comprises the administration of bacterial strains and/or derivatives thereof belonging to the same genus and/or species, for the improvement of the state of health, differentiated for male human subjects and female human subjects.

In the experimental section, as support and confirmation of the results surprisingly found, the Applicant has reported, by way of non-limiting example, an in vivo study conducted in humans, on a group of male patients and on a group of female patients, regarding the same neurodegenerative pathology represented by Parkinson's disease,

It was surprisingly found, solely by way of non-limiting example of the pathology studied, that as regards the curative or preventive treatment of Parkinson's disease, the administration of some bacterial strains and/or derivatives thereof produce better effects on male human patients and that the administration of other bacterial strains, also belonging to the same genus and/or species, but having a different biotype, produces better effects on female human patients.

In other words, the Applicant has found that a same biotype (same bacterial strain belonging to a same species and thus to a same genus) behaves in differentiated manner in male human patients and female human patients, the pathology or disorder being equal. And that biotypes differing from one another (different bacterial strains belonging, however, to a same species and hence to the same genus) improve the state of health of male human patients and female human patients, the pathology or disorder being equal. In this manner, with the present invention it is possible to choose and select, in an advantageous manner, the best biotype (the bacterial genus and species being equal) to be used in a differentiated manner on male human patients and female human patients, for the same pathology or disorder.

These results are unexpected since there are no known studies, or hypotheses, which correlate the effects of bacterial strains, also those belonging to the same genus and/or species, but having a different biotype, to the gender of the treated subjects in the improvement of the state of health differentiated for male and female human subjects.

More specifically, there are no known studies, or hypotheses, which correlate the effects of bacterial strains, and/or derivatives thereof, also those belonging to the same genus and/or species, but having a different biotype, to the gender of the treated human subjects, in the treatment of a pathology defined, by way of non-limiting example, by a neurodegenerative or cardiovascular pathology, or by a pathology linked to an immune system deficit, or by a pathology linked to biological processes of physical aging and aging of the skin or cutis, or by a pathology linked to biological aging processes which lead to a progressive loss of memory and/or of the ability to concentrate, or by a pathology caused by oxidative stress, or by an autoimmune pathology, or by an inflammatory pathology, or by a pathology due to an altered intestinal permeability, and others.

The bacterial strains and/or derivatives thereof of the present invention are bacterial strains that may belong to the genera Lactobacillus, Bifidobacterium, Lactococcus and Streptococcus, which, because of their biological activity, determine a targeted and differentiated interaction in the treatment of a male human subject and a female human subject.

The Applicant has surprisingly found that advantageous results are obtained in the treatment of several pathologies with selected bacterial strains in male human subjects and with other bacterial strains in female human subjects, but this certainly does not mean that certain bacterial strains (or bacterial genera) are effective solely and exclusively in subjects of a given sex.

In a preferred embodiment, in the composition for use according to the present invention said bacterial strain and/or derivatives thereof is selected from among Lactobacillus acidophilus, Lactobacillus brevis, Lactobacillus casei, Lactobacillus delbrueckii, Lactobacillus rhamnosus, Lactobacillus crispatus, Lactobacillus plantarum, Lactobacillus fermentum, Lactobacillus gasseri, Lactobacillus reuteri, Lactobacillus salivarius, Streptococcus thermophilus, Bifidobacterium adolescentis, Bifidobacterium bifidum, Bifidobacterium lactis, Bifidobacterium breve, Bifidobacterium longum, Bifidobacterium infantis, Bifidobacterium catenulatum, Bifidobacterium pseudocatenulatum and mixtures thereof.

In a preferred embodiment of the present invention, the composition of the present invention is for use in a method for treating a pathology selected, by way of non-limiting example, from among: (i) a pathology due to an imbalance in the intestinal microbiota, (ii) a neurodegenerative pathology, (iii) a cardiovascular pathology, (iv) a pathology linked to an immune system deficit, (v) a pathology linked to biological processes of physical aging and aging of the skin or cutis, (vi) a pathology linked to biological aging processes which lead to a progressive loss of memory and/or of the ability to concentrate, (vii) a pathology caused by oxidative stress, (viii) an autoimmune pathology, (ix) an inflammatory pathology, (x) a pathology due to an altered intestinal permeability, and others.

In a preferred embodiment, ma without limitations, the subject matter of the present invention relates to a composition comprising a mixture which comprises or, alternatively, consists of at least one bacterial strain and/or derivatives thereof for use in a method for the curative and/or preventive treatment of a pathology or disorder selected from (i) to (x) in a male human subject and female human subject, wherein said treatment comprises the administration of said composition to said subject, and said at least one strain and/or derivatives thereof is selected from among Lactobacillus salivarius LS01 DSM 22775 (deposited by Probiotical S.p.A. with the DSMZ on 23.07.2009), Lactobacillus plantarum LP01 LMG P—21021 (deposited by Mofin Srl with the BCCM-LMG on 16.10.2001), Lactobacillus reuteri DLLRE 09 DSM 25685 (deposited by Probiotical S.p.A. with the DSMZ on 16.02.2012), Lactobacillus acidophilus LA 02 DSM 21717 (deposited by Probiotical S.p.A. with the DSMZ on 06.08.2008), and Lactobacillus rhamnosus LR 06 DSM 21981 (deposited by Probiotical S.p.A. with the DSMZ on 14.11.2008) if the subject is a male human being, or mixtures of two or three or four of said bacterial strains and/or derivatives thereof; or, alternatively, if the subject is a female human being, said at least one bacterial strain and/or derivatives thereof is selected from among Lactobacillus salivarius LS01 DSM 22775 (deposited by Probiotical S.p.A. with the DSMZ on 23.07.2009), Lactobacillus salivarius LS 07 DSM 29476 (deposited by Probiotical S.p.A. with the DSMZ on 09.10.2014), Lactobacillus salivarius LS 06 DSM 26037 (deposited by Probiotical S.p.A. with the DSMZ on 06.06.2012), Bifidobacterium lactis BS01 LMG P-21384 (deposited by Anidral S.r.l. with the BCCM LMG on 31.01.2002 and a mixture (DLBLSS), preferably in a 1:1:1:1:1 ratio, consisting of the strain Bifidobacterium longum DLBL 07 DSM 25669 (deposited by Probiotical S.p.A. with the DSMZ on 16.02.2012), Bifidobacterium longum DLBL 08 DSM 25670 (deposited by Probiotical S.p.A. with the DSMZ on 16.02.2012), Bifidobacterium longum DLBL 09 DSM 25671 (deposited by Probiotical S.p.A. with the DSMZ on 16.02.2012), Bifidobacterium longum DLBL 10 DSM 25672 (deposited by Probiotical S.p.A. with the DSMZ on 16.02.2012), Bifidobacterium longum DLBL 11 DSM 25673 (deposited by Probiotical S.p.A. with the DSMZ on 16.02.2012), or mixtures of two or three or four of said bacterial strains and/or derivatives thereof.

In a preferred embodiment, the subject matter of the present invention relates to a composition for use in a method for the curative and/or preventive treatment of a pathology or disorder selected in the group comprising or, alternatively, consisting of (i) to (x) in a male human subject, wherein said method of treatment comprises the administration of said composition to said subject, and said at least one strain and/or derivatives thereof is selected in the group comprising or, alternatively, consisting of Lactobacillus salivarius LS01 DSM 22775 (deposited by Probiotical S.p.A. with the DSMZ on 23.07.2009), Lactobacillus plantarum LP01 LMG P-21021 (deposited by Mofin Srl with the BCCM-LMG on 16.10.2001), Lactobacillus reuteri DLLRE 09 DSM 25685 (deposited by Probiotical S.p.A. with the DSMZ on 16.02.2012), Lactobacillus acidophilus LA 02 DSM 21717 (deposited by Probiotical S.p.A. with the DSMZ on 06.08.2008), Lactobacillus rhamnosus LR 06 DSM 21981 (deposited by Probiotical S.p.A. with the DSMZ on 14.11.2008) and mixtures of two or three or four of said bacterial strains and/or derivatives thereof.

In a preferred embodiment, the subject matter of the present invention relates to a composition for use in the method for the curative and/or preventive treatment of a pathology or disorder selected in the group comprising or, alternatively, consisting of (i) to (x) in a female human subject, wherein said at least one bacterial strain and/or derivatives thereof is selected in the group comprising or, alternatively, consisting of Lactobacillus salivarius LS01 DSM 22775 (deposited by Probiotical S.p.A. with the DSMZ on 23.07.2009), Lactobacillus salivarius LS 07 DSM 29476 (deposited by Probiotical S.p.A. with the DSMZ on 09.10.2014), Lactobacillus salivarius LS 06 DSM 26037 (deposited by Probiotical S.p.A. with the DSMZ on 06.06.2012), Bifidobacterium lactis BS01 LMG P-21384 (deposited by Anidral S.r.l. with the BCCM LMG on 31.01.2002), a DLBLSS mixture, preferably in a ratio of 1:1:1:1:1, consisting of the strains Bifidobacterium longum DLBL 07 DSM 25669 (deposited by Probiotical S.p.A. with the DSMZ on 16.02.2012), Bifidobacterium longum DLBL 08 DSM 25670 (deposited by Probiotical S.p.A. with the DSMZ on 16.02.2012), Bifidobacterium longum DLBL 09 DSM 25671 (deposited by Probiotical S.p.A. with the DSMZ on 16.02.2012), Bifidobacterium longum DLBL 10 DSM 25672 (deposited by Probiotical S.p.A. with the DSMZ on 16.02.2012), Bifidobacterium longum DLBL 11 DSM 25673 (deposited by Probiotical S.p.A. with the DSMZ on 16.02.2012); and mixtures of two or three or four of said bacterial strains and/or derivatives thereof.

Preferably, in the composition for use according to the invention, the subject is a male human subject and said bacterial strain and/or derivatives thereof is selected from the group comprising or, alternatively, consisting of Lactobacillus plantarum LP01 LMG P-21021 (deposited by Mofin Srl with the BCCM-LMG on 16.10.2001), Lactobacillus reuteri DLLRE 09 DSM 25685 (deposited by Probiotical S.p.A. with the DSMZ on 16.02.2012), or mixtures thereof; said composition being for use in a method for the curative and/or preventive treatment of a pathology or disorder selected from (i) to (x) in a male human subject.

Preferably, in the composition for use according to the invention, the subject is a female human subject and said bacterial strain and/or derivatives thereof is selected from the group comprising or, alternatively, consisting of Lactobacillus salivarius LS 07 DSM 29476 (deposited by Probiotical S.p.A. with the DSMZ on 09.10.2014) and/or a mixture of Bifidobacterium longum DLBL 07 DSM 25669 (deposited by Probiotical S.p.A. with the DSMZ on 16.02.2012), Bifidobacterium longum DLBL 08 DSM 25670 (deposited by Probiotical S.p.A. with the DSMZ on 16.02.2012), Bifidobacterium longum DLBL 09 DSM 25671 (deposited by Probiotical S.p.A. with the DSMZ on 16.02.2012), Bifidobacterium longum DLBL 10 DSM 25672 (deposited by Probiotical S.p.A. with the DSMZ on 16.02.2012), Bifidobacterium longum DLBL 11 DSM 25673 (deposited by Probiotical S.p.A. with the DSMZ on 16.02.2012), or mixtures of two or three or four of said bacterial strains and/or derivatives thereof; said composition being for use in a method for the curative and/or preventive treatment of a pathology or disorder selected from (i) to (x) in a female human subject.

Preferably, the composition according to the invention is orally administered.

Within the scope of the present invention, “prevention” of pathologies means therapy aimed at avoiding the onset of such a disease in a male or female human subject, also, but not only, as a complication or effect of a pre-existing pathological condition or disorder.

In one embodiment, the administration of the composition to the male or female human subject takes place orally, for example in the form of a pill, tablet, which may also be coated, a capsule, solution, suspension, syrup, food containing the probiotic bacteria, or in any other form known to the person skilled in the art.

It remains understood that, if the treatment according to the invention comprises the administration of more than one bacterial strain and/or derivatives thereof, said administration according to the invention can take place simultaneously, for example in a single formulation, or in a rapid sequence, for example with two or more formulations taken by the subject in any order, in a sequence closely spaced in time (e.g. within 1 to 30 minutes) in two distinct compositions.

The composition for use according to the present invention can comprise, in addition to a bacterial strain and/or derivatives thereof, at least one inert ingredient, such as at least one excipient among the ones commonly used and known to the person skilled in the art.

“Inert ingredient” means any substance, or combination of substances, auxiliary to the production of a pharmaceutical, dietary or nutraceutical form, which is to be found in the finished product and is not the active ingredient, although it can modify the stability, release or other characteristics thereof.

Non-limiting examples of such ingredients, as is known to the person skilled in the art of formulations in the pharmaceutical, nutraceutical or food industry, are excipients such as diluents, absorbents, lubricants, colourants, surfactants, antioxidants, sweeteners, binders, disaggregating agents and others.

In one embodiment, the composition for use according to the present invention comprises, in addition to one or more bacterial strains and/or derivatives thereof, at least one further active ingredient of natural or synthetic origin. Non-limiting examples of said compounds are vitamins, antioxidants, or vegetable substances and preparations (botanicals).

In the context of the present invention, the term composition is meant to include, for example, a composition in the form of a medical device, or a composition suitable for food or nutraceutical use, for example as a dietary supplement, or in the form of a pharmaceutical composition or a cosmetic composition.

The composition of the present invention can be solid, liquid or semisolid, for example as a suspension or gel, and can be in any form known the person skilled in the art of the food, pharmaceutical or nutraceutical formulations, such as, by way of non-limiting example, in the form of a capsule, tablet, or powder that is at least partially dissolvable in the mouth or water soluble, granules, pellets or microparticles optionally contained in a sachet or in a capsule or mini-tablet, a liquid or semisolid preparation, gel, suspension, solution, two-phase liquid system and equivalent forms.

The following experimental part provides examples of practical embodiments of the invention, without limiting the scope thereof. In particular, the data provided below demonstrate, by way of non-limiting example, the fact that, for a specific pathology, some bacterial strains and/or derivatives thereof have demonstrated to possess particularly advantageous effects on male human subjects and other bacterial strains and/or derivatives thereof have demonstrated particularly advantageous effects on female human subjects. However, the strains that have demonstrated particular activity in male human subjects in the examples given below can be more active in female human subjects for different pathologies, and vice-versa.

Further embodiments are the following RPs:

RP1. A composition comprising a mixture which comprises, or consists of, an effective amount of at least one bacterial strain and/or at least one derivative thereof for use in the curative and/or preventive treatment, in a subject, of the symptoms and/or disorders connected to at least one pathology selected from among (i) a pathology due to an imbalance in the intestinal microbiota, (ii) a neurodegenerative pathology, (iii) a cardiovascular pathology, (iv) a pathology linked to an immune system deficit, (v) a pathology linked to biological processes of physical aging and of the skin or cutis, (vi) a pathology linked to biological aging processes which lead to a progressive loss of memory and/or of the ability to concentrate, (vii) a pathology caused by oxidative stress, (viii) an autoimmune pathology, (ix) an inflammatory pathology, (x) a pathology due to an altered intestinal permeability, and others, wherein said at least one bacterial strain preferably belongs to the genus Lactobacillus, Bifidobacterium, Lactococcus, Streptococcus, and wherein the bacterial strain performs a targeted and differentiated activity in the treatment of a male subject and a female subject.

RP2. The composition for use according to RP1, wherein the at least one bacterial strain and/or derivatives thereof is selected from among Lactobacillus acidophilus, Lactobacillus brevis, Lactobacillus casei, Lactobacillus delbrueckii, Lactobacillus rhamnosus, Lactobacillus crispatus, Lactobacillus plantarum, Lactobacillus fermentum, Lactobacillus gasseri, Lactobacillus reuteri, Lactobacillus salivarius, Streptococcus thermophilus, Bifidobacterium adolescentis, Bifidobacterium bifidum, Bifidobacterium lactis, Bifidobacterium breve, Bifidobacterium longum, Bifidobacterium infantis, Bifidobacterium catenulatum, Bifidobacterium pseudocatenulatum, and mixtures thereof.

RP3. The composition for use according to RP2, for use in the curative and/or preventive treatment of a pathology or disorder selected from (i) to (x) in a male human subject and female human subject, wherein said treatment comprises the administration of said composition to said subject, and said at least one strain and/or derivatives thereof is selected from among Lactobacillus salivarius LS01 DSM 22775 (deposited by Probiotical S.p.A. with the DSMZ on 23.07.2009), Lactobacillus plantarum LP01 LMG P-21021 (deposited by Mofin Srl with the BCCM-LMG on 16.10.2001), Lactobacillus reuteri DLLRE 09 DSM 25685 (deposited by Probiotical S.p.A. with the DSMZ on 16.02.2012), Lactobacillus acidophilus LA 02 DSM 21717 (deposited by Probiotical S.p.A. with the DSMZ on 06.08.2008), LR 06 DSM 21981 (deposited by Probiotical S.p.A. with the DSMZ on 14.11.2008) if the subject is a male human being, or mixtures of two or three or four of said bacterial strains and/or derivatives thereof;

or, alternatively, if the subject is a female human being, said at least one bacterial strain and/or derivatives thereof is selected from among Lactobacillus salivarius LS01 DSM 22775 (deposited by Probiotical S.p.A. with the DSMZ on 23.07.2009), Lactobacillus salivarius LS 07 DSM 29476 (deposited by Probiotical S.p.A. with the DSMZ on 09.10.2014), Lactobacillus salivarius LS 06 DSM 26037 (deposited by Probiotical S.p.A. with the DSMZ on 06.06.2012), Bifidobacterium lactis BS01 LMG P-21384 (deposited by Anidral S.r.l. with the BCCM LMG on 31.01.2002) and a mixture (DLBLSS), preferably 1:1:1:1:1, consisting of the strain Bifidobacterium longum DLBL 07 DSM 25669 (deposited by Probiotical S.p.A. with the DSMZ on 16.02.2012), Bifidobacterium longum DLBL 08 DSM 25670 (deposited by Probiotical S.p.A. with the DSMZ on 16.02.2012), Bifidobacterium longum DLBL 09 DSM 25671 (deposited by Probiotical S.p.A. with the DSMZ on 16.02.2012), Bifidobacterium longum DLBL 10 DSM 25672 (deposited by Probiotical S.p.A. with the DSMZ on 16.02.2012), Bifidobacterium longum DLBL 11 DSM 25673 (deposited by Probiotical S.p.A. with the DSMZ on 16.02.2012), or mixtures of two or three or four of said bacterial strains and/or derivatives thereof.

RP4. The composition for use according to RP3, wherein the subject is a male human subject and said bacterial strain and/or derivatives thereof is selected from the group comprising or, alternatively, consisting of Lactobacillus plantarum LP01 LMG P-21021 (deposited by Mofin Srl with the BCCM-LMG on 16.10.2001), Lactobacillus reuteri DLLRE 09 DSM 25685 (deposited by Probiotical S.p.A. with the DSMZ on 16.02.2012), or mixtures thereof; said composition being for use in the curative and/or preventive treatment of a pathology or disorder selected from (i) to (x) in a male human subject.

RP5. The composition for use according to RP3, wherein the subject is a female human subject and said bacterial strain and/or derivatives thereof is selected from the group comprising or, alternatively, consisting of Lactobacillus salivarius LS07 DSM 29476 (deposited by Probiotical S.p.A. with the DSMZ0 on 9.10.2014) and/or a mixture (DLBLSS), preferably 1:1:1:1:1, consisting of the strain Bifidobacterium longum DLBL 07 DSM 25669 (deposited by Probiotical S.p.A. with the DSMZ on 16.02.2012), Bifidobacterium longum DLBL 08 DSM 25670 (deposited by Probiotical S.p.A. with the DSMZ on 16.02.2012), Bifidobacterium longum DLBL 09 DSM 25671 (deposited by Probiotical S.p.A. with the DSMZ on 16.02.2012), Bifidobacterium longum DLBL 10 DSM 25672 (deposited by Probiotical S.p.A. with the DSMZ on 16.02.2012), Bifidobacterium longum DLBL 11 DSM 25673 (deposited by Probiotical S.p.A. with the DSMZ on 16.02.2012), or mixtures of two or three or four of said bacterial strains and/or derivatives thereof; said composition being for use in the curative and/or preventive treatment of a pathology or disorder selected from (i) to (x) in a female human subject.

RP6. The composition for use according to one of the preceding RPs, wherein the composition is administered orally.

### Experimental Part

The present in vitro study, conducted in humans (in vivo) by the inventors, aimed to analyse the activity of different bacterial strains and/or derivatives thereof on PBMCs (Peripheral Blood Mononuclear Cells) isolated from the blood of patients affected by Parkinson's disease in order to assess the direct effects of the microorganisms on oxidative stress and on the release of the cytokines most involved in the pathology. The inventors conducted the present in vitro study also to analyse the effects of the bacterial strain Lactobacillus salivarius LS03 (DSM 22776) vis-à-vis the other selected bacterial strains. The comparison was conducted on the main cellular subpopulations involved in the innate and acquired immune responses. Said subpopulations were isolated from the peripheral blood of subjects with Parkinson's disease, and were compared to those of healthy subjects.

TABLE 1

| STUDY STRAINS | Number - and deposit date | pro-Th2 | anti-IL17A |
|---|---|---|---|
| Lactobacillus acidophilus LA02 | DSM 21717 - Aug. 6, 2008 | X | X |
| L. delbrueckii subsp delbrueckii LDD01 | DSM 22106 - Dec. 10, 2008 | X | X |
| Lactobacillus plantarum LP01 | LMG P-21021 - Oct. 16, 2001 | X | X |
| Lactobacillus plantarum LP02 | LMG P-21020 - Oct. 16, 2001 | X | |
| Lactobacillus rhamnosus LR06 | DSM 21981 - Nov. 14, 2008 | X | |
| Lactobacillus reuteri LRE04 | DSM 23880 - Aug. 5, 2010 | X | X |
| Lactobacillus reuteri DLLRE09 | DSM 25685 - Feb. 16, 2012 | X | X |
| Lactobacillus salivarius LS01 | DSM 22775 - Jul. 23, 2009 | X | X |
| Lactobacillus salivarius LS02 | DSM 32204 - Nov. 13, 2015 | X | X |
| Lactobacillus salivarius LS03 | DSM 22776 - Jul. 23, 2009 | X | X |
| Lactobacillus salivarius LS06 | DSM 26037 - Jun. 6, 2012 | X | X |
| Lactobacillus salivarius LS07 | DSM 29476 - Oct. 9, 2014 | X | |
| GG | 1736 | X | |
| Bifidobacterium breve BR03 | DSM 16604 Jul. 20, 2004 | X | |
| Bifidobacterium breve BR05 | DSM 29494 Oct. 9, 2014 | X | |
| Bifidobacterium breve BS01 | LMG P-21384 Jan. 31, 2002 | X | |
| DLBL mixture of 5 strains (DLBL5S) | Bifidobacterium longum DLBL 07 | X | |
| | DSM 25669, Bifidobacterium | | |
| | longum DLBL 08 DSM 25670, | | |
| | Bifidobacterium longum DLBL 09 | | |
| | DSM 25671, Bifidobacterium | | |
| | longum DLBL 10 DSM 25672, | | |
| | Bifidobacterium longum DLBL 11 | | |
| | DSM 25673, all deposited on | | |
| | Feb. 16, 2012 | | |

Assessment of the implications of the action of the microorganisms on oxidative stress and on the modulation of the cytokine pattern in an in vitro model of PBMCs isolated from patients with Parkinson's disease.

### Materials and Methods

Bacterial strains and/or derivatives thereof: see Table 1

Subjects affected by Parkinson's disease (PD):
15 females and 25 males;
mean age: 69.9±8 years
27 patients undergoing pharmacological treatment with Levodopa (17 MALES/10 FEMALES)
13 patients without pharmacological treatment (8 MALES/5 FEMALES)
mean duration of the disease: 5±4 years; a 20 cc sample of venous blood was taken from all subjects.
the blood was mixed with heparin, as an anticoagulant, and processed on the same day it was collected.

The Peripheral Blood Mononuclear Cells (PBMCs) were isolated from the peripheral blood of Parkinson's subjects using the standard protocol of dextran sedimentation and Histopaque gradient centrifugation.

In this study, use was made of all the bacterial strains of the Probiotical collection in Table 1, which were cultured at T=37° C. in the selective medium MRS (De Man, Rogosa and Sharpe medium) for Lactobacilli and MRS+0.05% cysteine (by weight/total weight) for Bifidobacteria.

In the cellular model of PBMCs, the in vitro effects of the microorganisms on the production of the oxygen free radicals were assessed by means of an indirect spectrophotometric technique, the Superoxide Anion Assay (ROS test).

The modulation of the in vitro release of the most important pro- and anti-inflammatory cytokines by the PBMCs after incubation with the different bacterial strains and/or derivatives thereof under examination was also investigated, and the (Th1/Th2) ratio between the two cytokine families was subsequently assessed.

The data analysis and statistical assessment were performed with the ANOVA test; the data were considered significant for values of p<0.05.

### Results

### Assessment of Oxidative Stress

The monocytes (1×10⁶ cells/well) of Parkinson's patients were treated for 24 hours with the bacterial strains in Table 1. An indirect spectrophotometric test was then used to assess the reduction in Cytochrome C, which is directly proportional to the production of the superoxide anion (O₂⁻), an indicator of oxidative stress (FIG. 1); FIGS. 2 and 3 (§=indicator of very high activity) show the difference in the activity of the different bacterial strains and/or derivatives thereof tested on PBMCs originating from male patients (men) and female patients (women).

### Assessment of the Release of the Cytokines Th1 (Pro-Inflammatory) and Th2 (Anti-Inflammatory)

The PBMCs isolated from the blood of Parkinson's patients were stimulated for 24 hours with the strains (incubation at 37° C. and 5% 00₂). The release of the pro-inflammatory cytokine TNF-alpha (pro-Th1) and of the anti-inflammatory cytokine IL-10 (pro-Th2) was assessed. The release values obtained were used to calculate a Th1/Th2 ratio which indicated the activity of the strains on pro- or anti-inflammatory modulation (FIG. 4, ANOVA Test p<0.0001****, NS=not significant).

FIGS. 5 and 6 (§=indicator of very high activity) show the difference in the activity of the different microorganisms on PBMCs originating from male patients and female patients.

Conclusions: 12 bacterial strains out of 17 modulate the release of pro- and anti-inflammatory cytokines in a significant manner.

### Statistical Analysis of the Results

In Table in FIG. 7, the strains are listed in order from the “strongest” to the “weakest” (direction of the arrow); the statistical analysis (regression) was corrected for age parameters, duration of disease, UPDRS (Unified Parkinson's Disease Rating Scale) and treatment with Levodopa.

The bacterial strains tested for males (M) and females (F) (together and separate) were selected:

for their activity on the production of ROS (part 1 of the table in FIG. 7);

for their activity on cytokines (TH) (part 2 of the table in FIG. 7).

Combining the overall distance both of ROS and TH from neutrality (ROS=1 and TH=1), the tested bacterial strains with a greater effect on both activities were then selected (part 3 of the table).

1. A composition comprising a mixture which comprises or, alternatively, consists of an effective amount of at least one bacterial strain and/or at least one derivative thereof for use in a method for the curative and/or preventive treatment, in a subject, of the symptoms and/or disorders connected to at least one pathology selected in the group comprising or, alternatively, consisting of (i) a pathology due to an imbalance in the intestinal microbiota, (ii) a neurodegenerative pathology, (iii) a cardiovascular pathology, (iv) a pathology linked to an immune system deficit, (v) a pathology linked to biological processes of physical aging and aging of the skin or cutis, (vi) a pathology linked to biological aging processes which lead to a progressive loss of memory and/or of the ability to concentrate, (vii) a pathology caused by oxidative stress, (viii) an autoimmune pathology, (ix) an inflammatory pathology and (x) a pathology due to an altered intestinal permeability, wherein said at least one bacterial strain belongs to the genus selected in the group comprising or, alternatively, consisting of Lactobacillus, Bifidobacterium, Lactococcus and Streptococcus, wherein the bacterial strain performs a targeted and differentiated activity in the method of treatment for a male human subject and/or a female human subject, wherein said composition is for use in a method of treatment for a male human subject or, alternatively, a female human subject and wherein said subjects are affected by a pathology selected in the group comprising or, alternatively, consisting of (i) to (x).

2. The composition for use according to claim 1, wherein the at least one bacterial strain and/or derivatives thereof belongs to a species selected in the group comprising or, alternatively, consisting of Lactobacillus acidophilus, Lactobacillus brevis, Lactobacillus casei, Lactobacillus delbrueckii, Lactobacillus rhamnosus, Lactobacillus crispatus, Lactobacillus plantarum, Lactobacillus fermentum, Lactobacillus gasseri, Lactobacillus reuteri, Lactobacillus salivarius, Streptococcus thermophilus, Bifidobacterium adolescentis, Bifidobacterium bifidum, Bifidobacterium lactis, Bifidobacterium breve, Bifidobacterium longum, Bifidobacterium infantis, Bifidobacterium catenulatum, Bifidobacterium pseudocatenulatum, and mixtures thereof.

3. The composition for use according to claim 2, wherein said composition is for use in the method for the curative and/or preventive treatment of a pathology or disorder selected in the group comprising or, alternatively, consisting of (i) to (x) in a male human subject, wherein said method of treatment comprises the administration of said composition to said subject, and said at least one strain and/or derivatives thereof is selected in the group comprising or, alternatively, consisting of Lactobacillus salivarius LS01 DSM 22775 deposited by Probiotical S.p.A. with the DSMZ on 23.07.2009, Lactobacillus plantarum LP01 LMG P-21021 deposited by Mofin Srl with the BCCM-LMG on 16.10.2001, Lactobacillus reuteri DLLRE 09 DSM 25685 deposited by Probiotical S.p.A. with the DSMZ on 16.02.2012, Lactobacillus acidophilus LA 02 DSM 21717 deposited by Probiotical S.p.A. with the DSMZ on 06.08.2008, Lactobacillus rhamnosus LR 06 DSM 21981 deposited by Probiotical S.p.A. with the DSMZ on 14.11.2008 and mixtures of two or three or four of said bacterial strains and/or derivatives thereof.

4. The composition for use according to claim 2, wherein said composition is for use in the method for the curative and/or preventive treatment of a pathology or disorder selected in the group comprising or, alternatively, consisting of (i) to (x) in a female human subject, wherein said at least one bacterial strain and/or derivatives thereof is selected in the group comprising or, alternatively, consisting of Lactobacillus salivarius LS01 DSM 22775 deposited by Probiotical S.p.A. with the DSMZ on 23.07.2009, Lactobacillus salivarius LS 07 DSM 29476 deposited by Probiotical S.p.A. with the DSMZ on 09.10.2014, Lactobacillus salivarius LS 06 DSM 26037 deposited by Probiotical S.p.A. with the DSMZ on 06.06.2012, Bifidobacterium lactis BS01 LMG P-21384 deposited by Anidral S.r.l. with the BCCM LMG on 31.01.2002, a mixture DLBLSS, preferably in a ratio of 1:1:1:1:1, consisting of the strains Bifidobacterium longum DLBL 07 DSM 25669 deposited by Probiotical S.p.A. with the DSMZ on 16.02.2012, Bifidobacterium longum DLBL 08 DSM 25670 deposited by Probiotical S.p.A. with the DSMZ on 16.02.2012, Bifidobacterium longum DLBL 09 DSM 25671 deposited by Probiotical S.p.A. with the DSMZ on 16.02.2012, Bifidobacterium longum DLBL 10 DSM 25672 deposited by Probiotical S.p.A. with the DSMZ on 16.02.2012, Bifidobacterium longum DLBL 11 DSM 25673 deposited by Probiotical S.p.A. with the DSMZ on 16.02.2012; and mixtures of two or three or four of said bacterial strains and/or derivatives thereof.

5. The composition for use according to claim 3, wherein the subject is a male human subject and said bacterial strain and/or derivatives thereof is selected from the group comprising or, alternatively, consisting of Lactobacillus plantarum LP01 LMG P-21021 deposited by Mofin Srl with the BCCM-LMG on 16.10.2001, Lactobacillus reuteri DLLRE 09 DSM 25685 deposited by Probiotical S.p.A. with the DSMZ on 16.02.2012 and mixtures thereof; said composition being for use in a method for the curative and/or preventive treatment of a pathology or disorder selected in the group comprising or, alternatively, consisting of (i) to (x) in a male human subject.

6. The composition for use according to claim 4, wherein the subject is a female human subject and said bacterial strain and/or derivatives thereof is selected from the group comprising or, alternatively, consisting di: Lactobacillus salivarius LS07 DSM 29476 deposited by Probiotical S.p.A. with the DSMZ on 09.10.2014, a mixture (DLBLSS), preferably in a ratio of 1:1:1:1:1, consisting of the strains Bifidobacterium longum DLBL 07 DSM 25669 deposited by Probiotical S.p.A. with the DSMZ on 16.02.2012, Bifidobacterium longum DLBL 08 DSM 25670 deposited by Probiotical S.p.A. with the DSMZ on 16.02.2012, Bifidobacterium longum DLBL 09 DSM 25671 deposited by Probiotical S.p.A. with the DSMZ on 16.02.2012, Bifidobacterium longum DLBL 10 DSM 25672 deposited by Probiotical S.p.A. with the DSMZ on 16.02.2012, Bifidobacterium longum DLBL 11 DSM 25673 deposited by Probiotical S.p.A. with the DSMZ on 16.02.2012, and mixtures thereof; said composition being for use in a method for the curative and/or preventive treatment of a pathology or disorder selected in the group comprising or, alternatively, consisting of (i) to (x) in a female human subject.

7. The composition for use according to any one of the preceding claims, wherein the composition is administered orally.
